(19) United States
(12) Patent Application Publication
(10) Pub. No.: US 2015/0251973 A1
(43) Pub. Date: Sep. 10, 2015

(54) Purge Streams in Paraxylene Production

(71) Applicant: ExxonMobil Chemical Patents Inc., Baytown, TX (US)

(72) Inventors: Robert G. Tinger, Friendswood, TX (US); John Di-Yi Ou, Houston, TX (US)

(21) Appl. No.: 14/425,563

(22) PCT Filed: Sep. 6, 2013
(86) PCT No.: PCT/US2013/058526

§ 371 Date: Mar. 3, 2015

Related U.S. Application Data
(60) Provisional application No. 61/711,341, filed on Oct. 9, 2012.

(51) Int. Cl. C07C 5/27; C07C 4/18; C07C 2/86

(52) U.S. Cl. CPC C07C 5/2737; C07C 2/865; C07C 2/864; C07C 5/2775; C07C 4/18; C07C 2529/06

(57) ABSTRACT

In a process for producing para-xylene, a hydrocarbon feed comprising xylenes and ethylbenzene is supplied to a para-xylene extraction system, where a para-xylene-rich stream is recovered from the feed to leave at least one para-xylene-depleted stream. At least a portion of the para-xylene-depleted stream is isomerized at least partially in the liquid phase to produce an isomerized stream having a higher para-xylene concentration than the para-xylene-depleted stream. At least a portion of the isomerized stream is then recycled to the para-xylene extraction system. To control ethylbenzene build-up in the process, a purge stream is removed from the para-xylene-depleted stream and/or the isomerized stream and is subjected to one or more chemical processing steps to produce benzene and/or para-xylene.

### CROSS-REFERENCE TO RELATED APPLICATIONS

This application claims the benefit of Provisional Application No. 61/711,341 filed Oct. 9, 2012, the disclosure of which is incorporated by reference in its entirety.

### FIELD

This application relates to processing purge streams from para-xylene production processes.

### BACKGROUND

Ethylbenzene (EB), para-xylene (PX), ortho-xylene (OX) and meta-xylene (MX) are often present together in C₈ aromatic product streams from chemical plants and oil refineries. Of these C₈ compounds, although EB is an important raw material for the production of styrene, for a variety of reasons most EB feedstocks used in styrene production are produced by alkylation of benzene with ethylene, rather than by recovery from a C₈ aromatics stream. Of the three xylene isomers, PX has the largest commercial market and is used primarily for manufacturing terephthalic acid and terephthalate esters for use in the production of various polymers such as poly(ethylene terephthalate), poly(propylene terephthalate), and poly(butene terephthalate). While OX and MX are useful as solvents and raw materials for making products such as phthalic anhydride and isophthalic acid, market demand for OX and MX and their downstream derivatives is much smaller than that for PX.

Given the higher demand for PX as compared with its other isomers, there is significant commercial interest in maximizing PX production from any given source of C₈ aromatic materials. However, there are a number of major technical challenges to be overcome in achieving this goal of maximizing PX yield. For example, the four C₈ aromatic compounds, particularly the three xylene isomers, are usually present in concentrations dictated by the thermodynamics of production of the C₈ aromatic stream in a particular plant or refinery. As a result, the PX production is limited, at most, to the amount originally present in the C₈ aromatic stream unless additional processing steps are used to increase the amount of PX and/or to improve the PX recovery efficiency.

A variety of methods are known to increase the concentration of PX in a C₈ aromatics stream. These methods normally involve cycling the stream between a separation step, in which at least part of the PX is recovered to produce a PX-depleted stream, and a xylene isomerization step, in which the PX content of the PX-depleted stream is returned back towards equilibrium concentration. One commercially advantaged method of increasing PX yield involves conducting the xylene isomerization under at least partially liquid phase conditions so as to minimize xylene loss. However, under these conditions, little or none of the EB may be converted in the xylene isomerization step and as a result the amount of EB in the xylenes loop can build up to very high levels. Thus, to control the concentration of EB in the xylenes loop, it is frequently necessary to remove a purge stream from the xylenes loop. Although also containing valuable xylenes, this purge stream is typically sent to the motor gasoline pool or other use that has a lower economic value than that of PX. The present application seeks to provide a process for upgrading the purge stream to convert it into more valuable products including benzene and/or PX.

### SUMMARY

According to the invention, there is provided a process for producing para-xylene, the process comprising:
(a) supplying a hydrocarbon feed comprising xylenes and ethylbenzene to a para-xylene extraction system;
(b) recovering a para-xylene-rich stream from said feed in said para-xylene extraction system to leave at least one para-xylene-depleted stream;
(c) isomerizing at least a portion of said para-xylene-depleted stream at least partially in the liquid phase to produce an isomerized stream having a higher para-xylene concentration than said para-xylene-depleted stream;
(d) recycling at least a portion of the isomerized stream to said para-xylene extraction system;
(e) removing a purge stream from said para-xylene-depleted stream and/or said isomerized stream; and
(f) subjecting at least a portion of said purge stream to at least one chemical processing step selected from:
   (i) isomerizing ethylbenzene to produce para-xylene;
   (ii) deethylating ethylbenzene to produce benzene;
   (iii) deethylating ethylbenzene to produce benzene and isomerizing xylenes to produce para-xylene;
   (iv) deethylating ethylbenzene to produce benzene and methylating said benzene to form toluene and para-xylene; and
   (v) methylating ethylbenzene to form methylated ethylbenzene and/or polymethylated ethylbenzene, deethylating said methylated ethylbenzene and/or said polymethylated ethylbenzene to form toluene and para-xylene and methylating said toluene to form para-xylene.

In one embodiment, the processing step comprises deethylating ethylbenzene at least partially in the vapor phase to produce a benzene-containing effluent stream having a lower ethylbenzene concentration than said para-xylene-depleted stream and/or said isomerized stream.

In another embodiment, the processing step comprises isomerizing ethylbenzene at least partially in the vapor phase to produce a benzene-containing effluent stream having a lower ethylbenzene concentration than said para-xylene-depleted stream and/or said isomerized stream.

In yet another embodiment, the processing step comprises deethylating ethylbenzene and isomerizing xylenes at least partially in the vapor phase to produce a benzene-containing effluent stream having a lower ethylbenzene concentration than said para-xylene-depleted stream and/or said isomerized stream.

Conveniently, the process further comprises recycling at least part of said effluent stream to said para-xylene extraction system. Optionally, said para-xylene extraction system is used to generate a benzene- and/or toluene-containing C₇− stream from said effluent stream. The benzene- and/or toluene-containing C₇− stream can then be reacted with methanol and/or dimethyl ether in a methylation reactor to form a para-xylene-containing product stream, which can be recycled to the para-xylene extraction system.

In one embodiment, the para-xylene extraction system comprises at least one distillation unit for removing C₇− and C₉+ components and a fractional crystallization unit or selective adsorption unit for recovering said para-xylene-rich stream.

In one embodiment, the hydrocarbon feed is produced by reacting benzene and/or toluene with methanol and/or dimethyl ether in a methylation reactor.

### BRIEF DESCRIPTION OF THE DRAWINGS

FIG. 1 is a flow diagram of a conventional para-xylene (PX) production and extraction process which employs liquid phase xylene isomerization and removal of a purge stream to control ethylbenzene (EB) build up.

FIG. 2 (a) is a flow diagram of a process according to the present invention wherein a purge stream is sent to a vapor phase xylene isomerization unit.

FIG. 2 (b) is a flow diagram of a process according to the present invention wherein a purge stream is sent to a methylation reactor for converting ethylbenzene to para-xylene.

FIG. 2 (c) is a flow diagram of a process according to the present invention wherein a purge stream is sent to an EB dealkylation unit and then to a methylation reactor.

FIG. 2 (d) is a flow diagram of a process according to the present invention wherein a purge stream is sent to a unit for isomerizing EB to PX.

### DETAILED DESCRIPTION OF THE DRAWINGS

As used herein the term “Cₙ” hydrocarbon, wherein n is a positive integer, means a hydrocarbon having n number of carbon atom(s) per molecule. For example, a C₈ aromatic hydrocarbon means an aromatic hydrocarbon or mixture of aromatic hydrocarbons having 8 number of carbon atom(s) per molecule. The term “Cₙ+” hydrocarbon, wherein n is a positive integer, means a hydrocarbon having at least n number of carbon atom(s) per molecule, whereas the term “Cₙ−” hydrocarbon wherein n is a positive integer, means a hydrocarbon having no more than n number of carbon atom(s) per molecule.

A process for producing PX comprises supplying a hydrocarbon feed comprising xylenes and EB to a PX extraction system, where a PX-rich stream is recovered from the feed to leave a PX-depleted stream. At least part of the PX-depleted stream is then fed to a xylene isomerization unit where the PX-depleted stream is isomerized under at least partial liquid phase conditions to produce an isomerized stream having a higher PX concentration than the

PX-depleted stream. At least part of the isomerized stream is then recycled to the PX extraction system to recover additional PX and the process is repeated to define a so-called xylene isomerization loop. Little or none of the ethylbenzene in the hydrocarbon feed is converted in the xylene isomerization step and hence, to avoid a build-up of EB in the xylene isomerization loop, a purge stream is removed from the PX-depleted stream and/or the isomerized stream. In the present process, rather than feeding this purge stream to the motor gasoline pool or other low value application, at least a portion of said purge stream is provided to one or more chemical processing steps for converting EB to advantaged chemicals, such as benzene and more particularly additional PX.

### Hydrocarbon Feed

The feed employed in the present process may be any hydrocarbon stream containing C₈ aromatic hydrocarbons, such as a reformate stream (product stream of a reformate splitting tower), a hydrocracking product stream, a xylene or EB reaction product stream, an aromatic alkylation product stream, an aromatic disproportionation stream, an aromatic transalkylation stream, and/or a Cyclar™ process stream.

In one embodiment, the feed is the product of the alkylation of benzene and/or toluene with methanol and/or dimethyl ether in a methylation reactor. One such methylation reactor is described in U.S. Pat. Nos. 6,423,879 and 6,504,072, the entire contents of which are incorporated herein by reference, and employs a catalyst comprising a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa). The porous crystalline material may be a medium-pore zeolite, such as ZSM-5, which has been severely steamed at a temperature of at least 950° C. in the presence of at least one oxide modifier, for example including phosphorus, to control reduction of the micropore volume of the material during the steaming step. Such a methylation reactor is hereinafter termed a “PX-selective methylation reactor”.

The feedstock may further comprise recycle stream(s) from the isomerization step(s) and/or various separating steps. The hydrocarbon feed comprises PX, together with meta-xylene (MX), ortho-xylene (OX), and/or EB. In addition to xylenes and EB, the hydrocarbon feedstock may also contain certain amounts of other aromatic or even non-aromatic compounds. Examples of such aromatic compounds are C₇− hydrocarbons, such as benzene and toluene, and C₉+ aromatics, such as mesitylene, pseudo-cumene and others. These types of feedstream(s) are described in “Handbook of Petroleum Refining Processes”, ds. Robert A. Meyers, McGraw-Hill Book Company, Second Edition.

### Para-Xylene Extraction

The hydrocarbon feed is initially supplied to a PX extraction system to recover a PX-rich product stream from the feed and leave a PX-depleted stream. In one embodiment, the PX-rich product stream comprises at least 50 wt % PX, preferably at least 60 wt % PX, more preferably at least 70 wt % PX, even preferably at least 80 wt % PX, still even preferably at least 90 wt % PX, and most preferably at least 95 wt % PX, based on the total weight of the PX rich product stream. The PX extraction system can include one or more of any of the PX recovery units known in the art, including, for example, a crystallization unit, an adsorption unit such as a PAREX™ unit or an ELUXYL™ unit, a reactive separation unit, a membrane separation unit, an extraction unit, a distillation unit, an extractive distillation unit, a fractionation unit, or any combination thereof These types of separation unit(s) and their designs are described in “Perry's Chemical Engineers' Handbook”, Eds. R. H. Perry, D. W. Green and J. O. Maloney, McGraw-Hill Book Company, Sixth Edition, 1984, and the previously-mentioned “Handbook of Petroleum Refining Processes”.

Depending on the composition of the hydrocarbon feed, the PX extraction system may include one or more initial separation steps that serve to remove C₇− and C₉+ hydrocarbons from the feed prior to extraction of the PX-rich product stream. Generally the initial separation steps may include fractional distillation, crystallization, adsorption, a reactive separation, a membrane separation, extraction, or any combination thereof

### Xylene Isomerization

After recovery of the PX-rich product stream, the remaining PX-depleted stream is fed to a xylene isomerization unit where the PX-depleted stream is contacted with a xylene isomerization catalyst under at least partially liquid phase conditions effective to isomerize the PX-depleted stream back towards an equilibrium concentration of the xylene isomers. Suitable conditions include a temperature of from about 400° F. (about 204° C.) to about 1,000° F. (about 538° C.), a pressure of from about 0 to 1,000 psig, a weight hourly space velocity (WHSV) of from 0.5 to 100 hr⁻¹, with the pressure and temperature being adjusted within the above ranges to ensure that at least part of the C₈ aromatics in the PX-depleted stream is in the liquid phase. Generally, the conditions are selected so that at least 50 wt % of the C₈ aromatics would be expected to be in the liquid phase.

Any catalyst capable of isomerizing xylenes in the liquid phase can be used in the xylene isomerization unit, but in one embodiment the catalyst comprises an intermediate pore size zeolite having a Constraint Index between 1 and 12. Constraint Index and its method of determination are described in U.S. Pat. No. 4,016,218, which is incorporated herein by reference. Particular examples of suitable intermediate pore size zeolites include ZSM-5, ZSM-11, ZSM-12, ZSM-22, ZSM-23, ZSM-35, ZSM-48, and MCM-22, with ZSM-5 and ZSM-11 being particularly preferred, specifically ZSM-5. It is preferred that the acidity of the zeolite, expressed as its alpha value, be greater than 300, such as greater than 500, or greater than 1000. The alpha test is described in U.S. Pat. No. 3,354,078; in the Journal of Catalysis, Vol. 4, p. 527 (1965); Vol. 6, p. 278 (1966); and Vol. 61, p. 395 (1980), each incorporated herein by reference as to that description. The experimental conditions of the test used to determine the alpha values cited herein include a constant temperature of 538° C. and a variable flow rate as described in detail in the Journal of Catalysis, Vol. 61, p. 395.

The product of the xylene isomerization process is an isomerized stream having a higher PX concentration than the PX-depleted stream. The isomerized stream is then recycled to the PX extraction system to recover additional PX and the process is repeated to generate a so-called xylene isomerization loop.

### Treatment of the Puree Stream

The catalyst and conditions in the xylene isomerization process are selected to minimize xylene transalkylation and other reactions leading to xylene loss. As a result the

EB in the PX-depleted feed to a xylene isomerization unit remains largely unconverted during the xylene isomerization process and hence, to avoid a build-up of EB in the xylene isomerization loop, a purge stream is removed from the PX-depleted stream and/or the isomerized stream. The purge stream can be removed continuously or intermittently from the isomerization loop. One of ordinary skill in the art, in possession of the present disclosure, can determine the amount of purge stream relative to the total material flow in the loop. The purge stream is fed to one or more chemical processing units, conveniently existing chemical processing units co-located with liquid phase xylene isomerization unit, to convert at least part of the EB in the purge stream to advantaged chemicals, such as benzene and more particularly additional PX.

Examples of suitable chemical processing steps include:
(i) isomerization of the EB in the purge stream to produce xylenes;
(ii) deethylation of the EB in the purge stream to produce benzene, optionally with simultaneous isomerization of the xylenes in the purge stream and further optionally with methylation of the benzene to produce toluene and additional PX; and
(v) methylation of the EB in the purge stream to form methylated and/or polymethylated ethylbenzene, followed by deethylation of the methylated and/or polymethylated ethylbenzene to form toluene and para-xylene and then methylation of the toluene to form additional PX.

It is well known that EB can be removed from a hydrocarbon stream by at least two competing reaction mechanisms, one by isomerization to xylenes and the other by dealkylation to benzene and C₂ light gas. In most cases, although the catalyst and conversion conditions may be selected to favor one reaction mechanism, the other mechanism will also occur to a varying degree. In addition, where the hydrocarbon stream contains non-equilibrium xylenes, such as when the purge stream is removed from the PX-depleted stream, the EB conversion will be accompanied by isomerization of the xylenes in the stream.

### Ethylbenzene Isomerization

Ethylbenzene, such as that contained in the present purge stream, may be isomerized to xylenes in the presence of an acid catalyst at sufficient temperature, pressure, and space velocity conditions, which can be determined by one of ordinary skill in the art in possession of the present disclosure, such as up to about 500° C., a pressure from about 10 kPa to about 5 MPa absolute, and a liquid hourly space velocity with respect to the hydrocarbon feed mixture of from about 0.1 to 30 hr ⁻¹. The hydrocarbon feed mixture optimally is reacted in admixture with hydrogen at a hydrogen/hydrocarbon mole ratio of about 0.5:1 to about 25:1. The isomerization conditions are selected so that the C₈ aromatics in the purge stream are at least partially in the vapor phase. Generally, the isomerization conditions are selected so that at least 50 wt %, and in one embodiment essentially all, of the C₈ aromatics would be expected to be in the vapor phase. Conditions should be adjusted so that EB isomerization is favored over EB dealkylation, which can be accomplished by routine experimentation by one of ordinary skill in the art in possession of the present disclosure.

The acid catalyst employed for the EB isomerization reaction is preferably an intermediate pore size zeolite, such as ZSM-5, ZSM-11, ZSM-12, ZSM-22, ZSM-23, ZSM-35, ZSM-48, and MCM-22, with ZSM-5 being particularly preferred. Catalyst properties that favor EB isomerization over EB dealkylation include a low acid activity, such as an alpha value less than 50, and an ortho-xylene sorption time less than, by way of example, 50 minutes, wherein ortho-xylene sorption time is the time required to sorb 30% of the equilibrium capacity of ortho-xylene at 120° C. and at an ortho-xylene partial pressure of 4.5+0.8 mm of mercury. This test is described in U.S. Patent Nos. 4,117,026; 4,159,282; and Re. 31,782. With intermediate pore size zeolites, the desired xylene diffusion properties may be achieved by the selection of a small crystal form (less than 0.5 micron) of the zeolite.

In one embodiment, where the hydrocarbon feed to the extraction system of the present process is produced by alkylation of benzene and/or toluene with methanol and/or dimethyl ether in a PX-selective methylation reactor, as defined above, isomerization of the EB contained in the purge stream may be effected by recycling the purge stream to the methylation reactor.

The product of EB isomerization of the purge stream is an effluent stream having a lower EB concentration and a higher xylene concentration, than the purge stream. The effluent stream may therefore be recycled to the PX extraction system to recover the additional PX.

### Ethylbenzene Dealkylation

The EB in the purge stream can be dealkylated to produce benzene using the same broad range of conditions and the same acid catalysts as discussed above in relation to EB isomerization.

Catalyst properties that favor EB dealkylation include a high acid activity, such as an alpha value of at least 50, typically from about 100 to about 500, and an ortho-xylene sorption time in excess of 50 minutes, typically greater than 1200 minutes, but less than 10,000 minutes. With intermediate pore size zeolites, the desired xylene diffusion properties may be achieved by the selection of a large crystal form (greater than 0.5 micron) of the zeolite and/or by pre-coking or silica selectivation of the zeolite as disclosed in U.S. Pat. No. 5,476,823, the entire contents of which are incorporated herein by reference.

The product of EB dealkylation of the purge stream is a benzene-containing effluent stream having a lower EB concentration than the purge stream. The benzene in the effluent stream can be recovered by, for example, recycling the effluent stream to an extraction system (e.g., a distillation system, such as discussed with reference to various figures, below, liquid-liquid extraction or extractive distillation systems, and the like) and sold. Alternatively, part or all of the benzene-containing effluent stream can be reacted methanol and/or dimethyl ether in a methylation reactor (again, as discussed below) to form a para-xylene-containing product stream. In one embodiment, the methylation reactor is a PX-selective methylation reactor, as defined above.

### Conversion of Ethylbenzene to Para-Xylene via Methylated Ethylbenzenes

In a further embodiment, the EB in the purge stream is converted by a three stage process involving initial reaction with methanol or dimethyl ether to form methylated and/or polymethylated ethylbenzenes (MEB), which are then deethylated to form an effluent stream containing toluene and PX. The PX can subsequently be recovered from the effluent stream, while the toluene can be methylated to form additional PX.

In one embodiment the three process stages of EB methylation, followed by MEB deethylation and then toluene methylation are conducted in separate reactors by methods well known in the art, whereas in other embodiments the three process stages are conducted in a single stage by, for example, supplying the EB-containing purge stream to a PX-selective methylation reactor, as defined above.

The invention will now be more particularly described with reference to the accompanying drawings, in which like numerals are used to identify like components in the different figures.

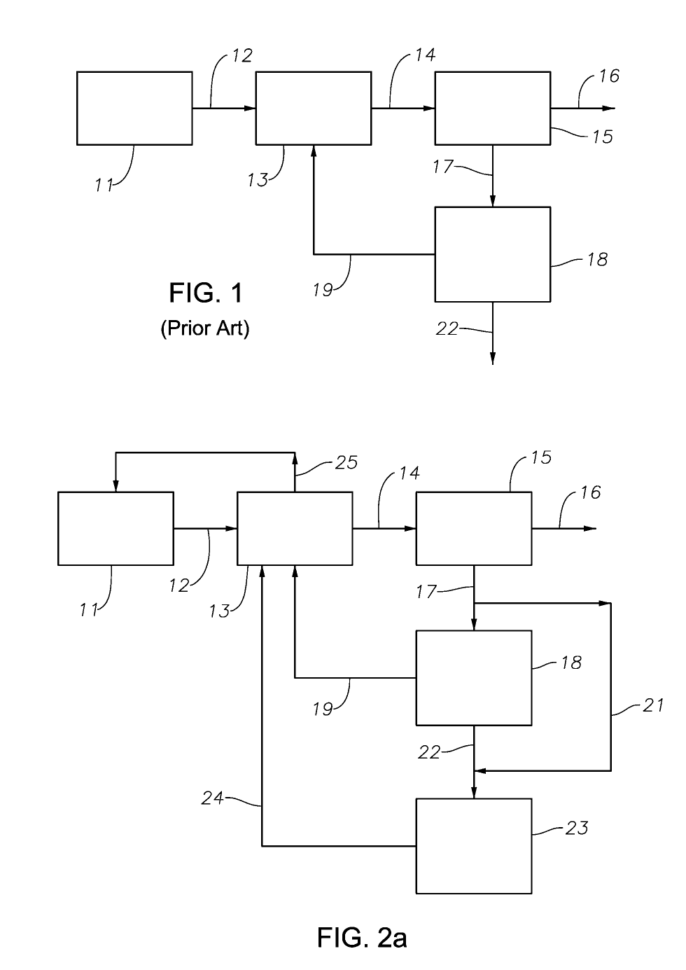

FIG. 1 is a flow diagram of a conventional PX production and extraction process, in which benzene and/or toluene is reacted with methanol and/or dimethyl ether in a PX-selective methylation reactor 11 to produce a hydrocarbon feed containing a mixture of C₈ aromatic hydrocarbons but also containing C₇− and C₉+ components. The hydrocarbon feed is supplied by line 12 to a distillation system 13, where the C₇− and C₉+ components are removed to leave a C₈-containing stream which is supplied by line 14 to a PX extraction unit 15. It will be appreciated by one of ordinary skill in the art that distillation system 13 in this and other figures described herein is illustrated by a single unit, but which may represent plural columns, such as one to take off C₇₋ species and one to take off C₉+ species. A PX-rich stream is recovered from the PX extraction unit 15 by way of line 16 to leave a PX-depleted stream, which is fed by line 17 to a liquid phase xylene isomerization unit 18. The unit 18 isomerizes the PX-depleted stream to produce an isomerized stream which has a higher PX concentration than the PX-xylene-depleted stream and which is recycled by line 19 to the distillation system 13.

In the process shown in FIG. 1 excessive build up of EB in the loop defined by the distillation system 13, the PX extraction unit 15 and the liquid phase xylene isomerization unit 18 is avoided by removing a purge stream from the unit 18 via line 22 and sending the purge stream to a gasoline blending station (not shown).

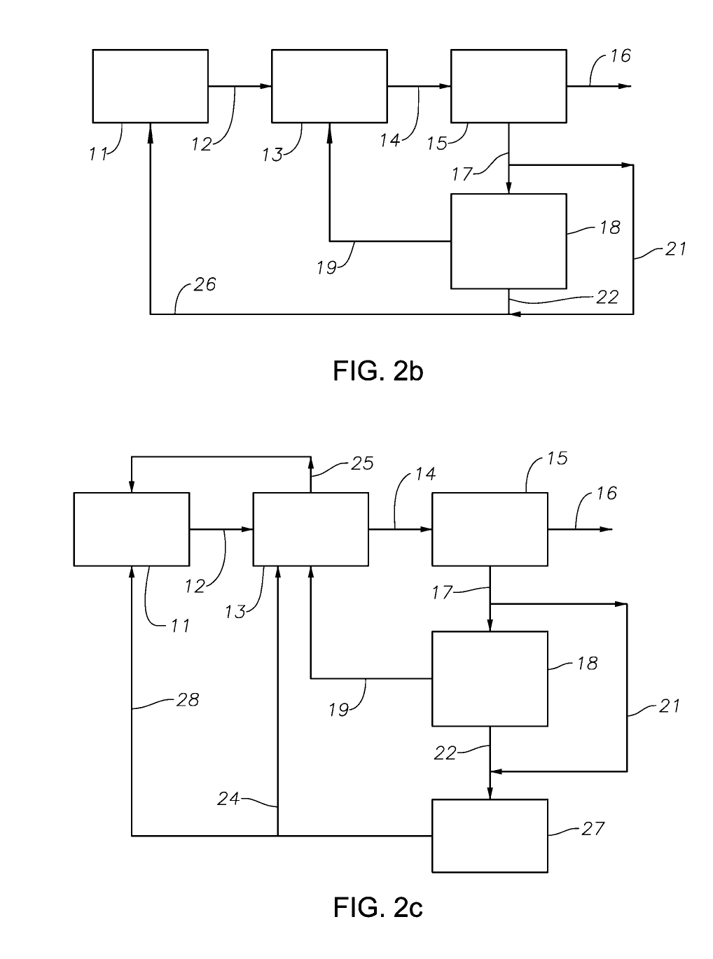

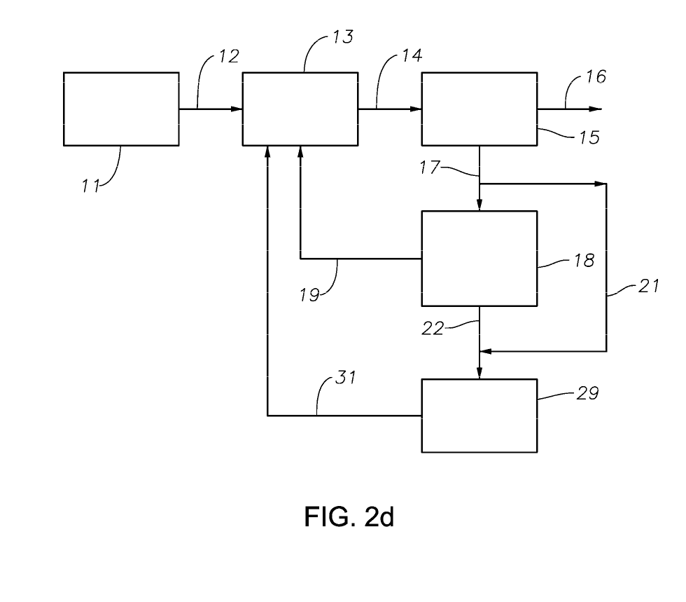

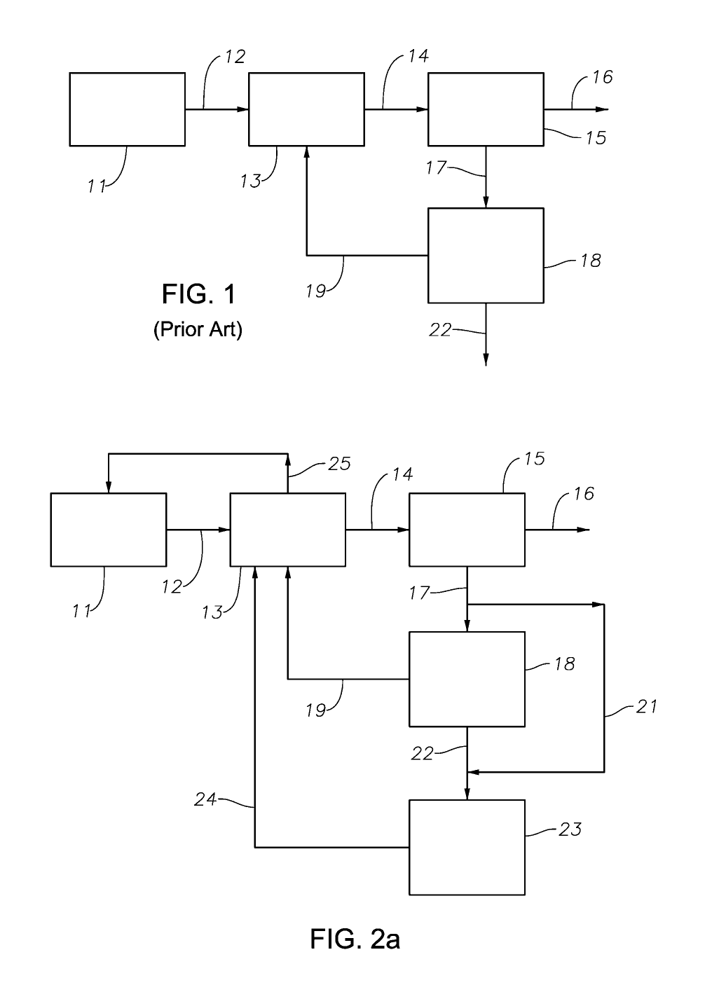

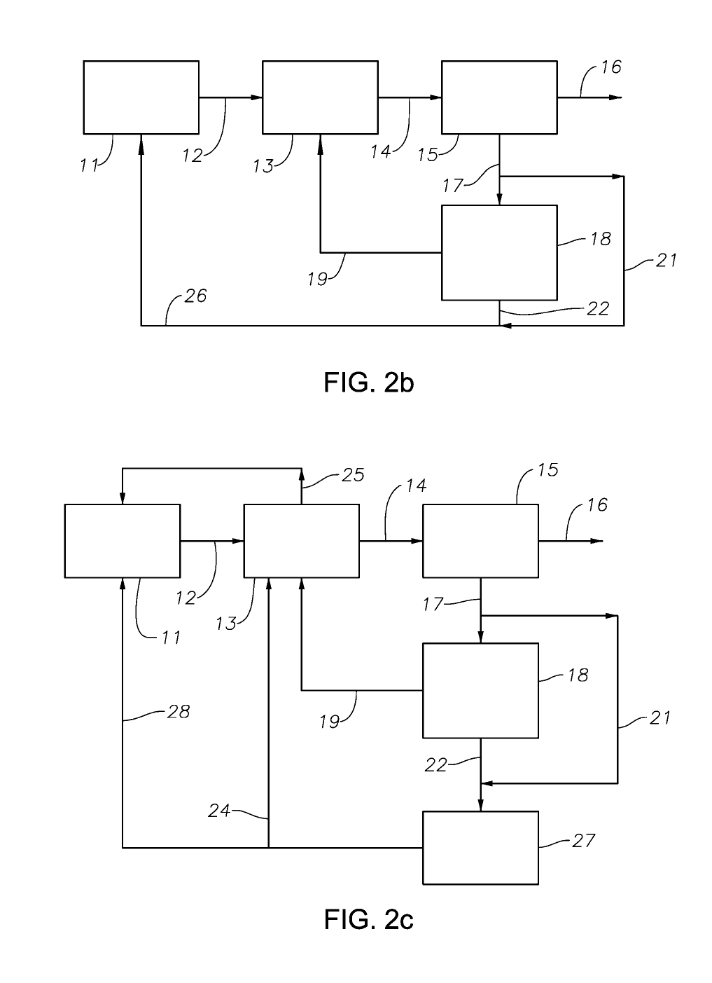

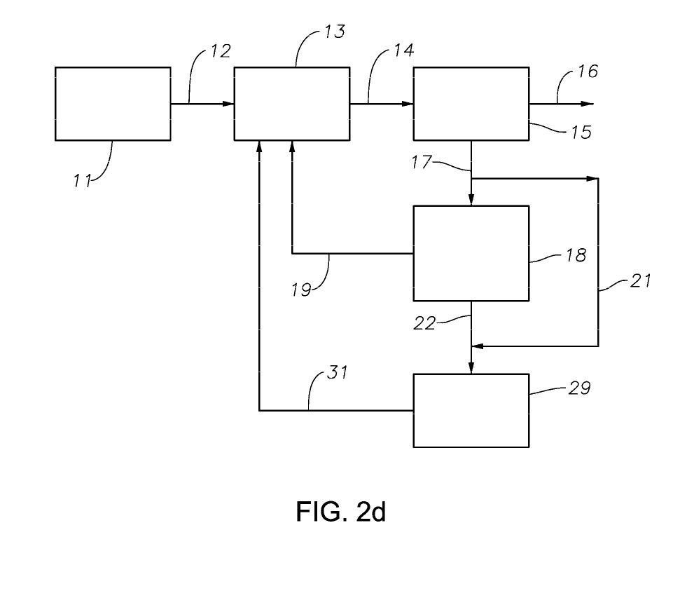

FIG. 2(a) illustrates a process according to a first embodiment of the present disclosure, in which a purge stream 21 is removed from the PX-depleted stream 17 (from PX extraction unit and/or from the liquid phase xylene isomerization unit 18 via line 22 and fed to a vapor phase isomerization unit 23. EB in the purge stream is dealkylated and xylenes in the purge stream are isomerized in the unit 23 to produce a benzene-containing effluent stream 24 having a lower ethylbenzene concentration and a higher PX concentration than the purge stream(s) M and/or 22. The benzene-containing effluent stream 24 is recycled to the distillation system 13, where the benzene is separated and fed by line 25 to the reactor 11 which, as in FIG. 1, produces a hydrocarbon feed containing a mixture of C₈ aromatic hydrocarbons supplied by line 12 to a distillation system 13, where, again, the C₇− and C₉+ components are removed to leave a C₈-containing stream which is supplied by line 14 to a PX extraction unit 15. A PX-rich stream is recovered from the PX extraction unit 15 by way of line 16 to leave a PX-depleted stream 17. A portion of the liquid phase isomerate is recycled via line 19 to distillation system 13.

FIG. 2(b) illustrates a process according to a second embodiment of the present disclosure, in which a purge stream 21 is removed from the PX-depleted stream 17 (from PX extraction unit 15) and/or purge stream 22 is removed from the liquid phase xylene isomerization unit 18, but in this second embodiment one or both purge streams are recycled by line 26 to the methylation reactor 11 where EB in the purge stream is isomerized to para-xylene. Distillation system 13 and fluid connections 12, 14, 17, and 19, and recovery of PX-enriched stream 16 are the same as in the first embodiment discussed with respect to FIG. 2a, above.

FIG. 2(c) illustrates a process according to a third embodiment of the present disclosure, in which a purge stream 21 is again removed from the paraxylene-depleted stream 17 and/or from the liquid phase xylene isomerization unit 18 via line 22, but in this third embodiment one or both purge streams are fed to an EB dealkylation unit 27. EB in the purge stream(s) 21 and/or 22 is deethylated in the EB dealkylation unit 27 to produce a benzene-containing effluent stream 28 having a lower ethylbenzene concentration than the purge stream. The benzene-containing effluent stream is either recycled to the methylation reactor 11 and/or recycled to distillation system 13 by line 24. Conduits 12, 14, 19, and 25 and paraxylene-enriched product recovery via line 16 are the same as discussed above with respect to the first and second embodiments.

FIG. 2(d) illustrates a process according to a fourth embodiment of the present disclosure, in which a purge stream 21 is removed from the PX depleted stream 17 and/or from the liquid phase xylene isomerization unit 18 via line 22, as in previous embodiments, and fed to a EB isomerization unit 29. EB in the purge stream is isomerized to xylenes in the vapor phase in the unit 29 to produce an effluent stream which has a lower ethylbenzene concentration than the purge stream and which is recycled via line 31 to the distillation system 13. Methylation unit 11, distillation system 13, paraxylene extraction unit 15, liquid phase isomerization unit 18, and associated fluid conduits 12, 14, 19, and paraxylene-enriched stream 16 recovery, as in previous embodiments, complete the fourth embodiment shown schematically in FIG. 2d.

While the present invention has been described and illustrated by reference to particular embodiments, those of ordinary skill in the art will appreciate that the invention lends itself to variations and modification is not necessarily illustrated herein without departing from the spirit and scope of the invention.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated.

1. A process for producing para-xylene, the process comprising:
(a) supplying a hydrocarbon feed comprising xylenes and ethylbenzene to a para-xylene extraction system;
(b) recovering a para-xylene-rich stream from said feed in said para-xylene extraction system to leave at least one para-xylene-depleted stream;
(c) isomerizing at least a portion of said para-xylene-depleted stream at least partially in the liquid phase to produce an isomerized stream having a higher para-xylene concentration than said para-xylene-depleted stream;
(d) recycling at least a portion of the isomerized stream to said para-xylene extraction system;
(e) removing a purge stream from said para-xylene-depleted stream and/or said isomerized stream; and
(f) subjecting at least a portion of said purge stream to at least one chemical processing step selected from:
(i) isomerizing ethylbenzene to produce para-xylene;
(ii) deethylating ethylbenzene to produce benzene;
(iii) deethylating ethylbenzene to produce benzene and isomerizing xylenes to produce para-xylene;
(iv) deethylating ethylbenzene to produce benzene and methylating said benzene to form toluene and para-xylene; and
(v) methylating ethylbenzene to form methylated ethylbenzene and/or polymethylated ethylbenzene, deethylating said methylated ethylbenzene and/or said polymethylated ethylbenzene to form toluene and para-xylene and methylating said toluene to form para-xylene.

2. The process of claim 1, wherein said processing step comprises isomerizing ethylbenzene at least partially in the vapor phase to produce an effluent stream having a lower ethylbenzene concentration than said para-xylene-depleted stream and/or said isomerized stream.

3. The process of claim 1, wherein said processing step comprises deethylating ethylbenzene at least partially in the vapor phase to produce a benzene-containing effluent stream having a lower ethylbenzene concentration than said para-xylene-depleted stream and/or said isomerized stream.

4. The process of claim 1, wherein said processing step comprises deethylating ethylbenzene and isomerizing xylenes at least partially in the vapor phase to produce a benzene-containing effluent stream having a lower ethylbenzene concentration than said para-xylene-depleted stream and/or said isomerized stream.

5. The process of claim 3 and further comprising reacting at least part of said benzene-containing effluent stream with methanol and/or dimethyl ether in a methylation reactor to form a para-xylene-containing product stream.

6. The process of claim 1 and further comprising recycling at least part of said para-xylene produced by step (f) to said para-xylene extraction system.

7. The process of claim 6 and further comprising recovering a benzene- and/or toluene-containing C₇− stream from said para-xylene extraction system.

8. The process of claim 7 and further comprising reacting at least part of said benzene- and/or toluene-containing C₇− stream with methanol and/or dimethyl ether in a methylation reactor to form a para-xylene-containing product stream.

9. (canceled)

10. The process of claim 5 and further comprising recycling at least part of said para-xylene-containing product stream to the para-xylene extraction system.

11. The process of claim 1, wherein said para-xylene extraction system comprises at least one distillation unit for removing C₇− and C₉+ components and a fractional crystallization unit or selective adsorption unit for recovering said para-xylene-rich stream.

12. The process of claim 1, wherein said hydrocarbon feed is produced by reacting benzene and/or toluene with methanol and/or dimethyl ether in a methylation reactor.

13. The process of claim 4 and further comprising reacting at least part of said benzene-containing effluent stream with methanol and/or dimethyl ether in a methylation reactor to form a para-xylene-containing product stream.

14. The process of claim 8 and further comprising recycling at least part of said para-xylene-containing product stream to the para-xylene extraction system.
